(19) United States
(12) Patent Application Publication
(10) Pub. No.: US 2009/0197917 A1
(43) Pub. Date: Aug. 6, 2009

(54) USE OF RIMONABANT FOR THE PREPARATION OF MEDICAMENTS USEFUL IN THE PREVENTION AND TREATMENT OF TYPE 2 DIABETES

(75) Inventors: Corinne HANOTIN, La Celle Saint Cloud (FR); Pierre Rosenzweig, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/402,988

(22) Filed: Mar. 12, 2009

Related U.S. Application Data
(63) Continuation of application No. 11/832,865, filed on Aug. 2, 2007.
Application No. 11/832,865 is a continuation of application No. PCT/FR2006/000376, filed on Feb. 20, 2006.

(30) Foreign Application Priority Data
Feb. 21, 2005 (FR) 0501861
May 12, 2005 (FR) 0504942
May 23, 2005 (FR) 0505228

(51) Int. Cl. A61K 31/454

(52) U.S. Cl. 514/326

(57) ABSTRACT

The invention relates to the use of rimonabant, either alone or combined with another active ingredient, for the preparation of medicaments useful in the prevention and treatment of type 2 diabetes or non-insulin-dependent diabetes and/or its complications.

This application is a continuation of U.S. application Ser. No. 11/832,865, filed Aug. 2, 2007, now pending, which is a continuation of International application No. PCT/FR2006/000,376, filed Feb. 20, 2006, both of which are incorporated herein by reference in their entirety; which claims the benefit of priority of French Patent Application No. 05/01,861, filed Feb. 21, 2005, French Patent Application No. 05/04,942, filed May 12, 2005 and French Patent Application No. 05/05,228, filed May 23, 2005.

The subject of the present invention is the use of rimonabant for the preparation of medicaments useful in the prevention and treatment of type 2 diabetes or non-insulin-dependent diabetes and/or its complications.

Type 2 diabetes is characterized by insulin-secretion disorders associated with insulin-sensitivity or insulin-resistance disorders. Insulin resistance is aggravated by hyperglycaemia and by high levels of circulating free fatty acids and of stored triglycerides.

Rimonabant is the international non-proprietary name for N-piperidino-5-(4-chlorophenyl)-1-(2,4-dichlorophenyl)-4-methylpyrazole-3-carboxamide described in European Patent 656354.

Clinical studies carried out with rimonabant have shown that it acts on food intake from the quantitative and qualitative point of view and reduces the body weight of obese patients (G. Le Fur, 2003, 35, First European Workshop on Cannabinoid Research, Madrid, Spain, 4-5 Apr. 2003 and Heshmati H. M. et al., Obesity Research, 2001, 9 (suppl. 3), 70.

It has now been found that rimonabant has antidiabetic properties and acts on complications linked to diabetes.

Thus, according to the present invention, rimonabant can be used for the preparation of medicaments useful for preventing and treating type 2 diabetes and its complications.

The expression complications linked to diabetes is understood to mean:
cardiovascular diseases linked to diabetes;
neurological diseases such as diabetic neuropathies, peripheral neuropathies, autonomous cardiac neuropathies;
renal diseases such as diabetic nephropathies, diabetic glomerulopathies;
ocular diseases such as diabetic retinopathies, macular oedemas, glaucoma;
angiopathies: microangiopathies, macroangiopathies, coronaropathies, peripheral arteriopathies.

According to one of its aspects, the subject of the present invention is the use of rimonabant for the prevention and treatment of the complications linked to diabetes, most particularly, peripheral neuropathies, diabetic nephropathies, diabetic retinopathies, angiopathies.

The pharmaceutical compositions according to the present invention contain an effective dose of rimonabant and at least one pharmaceutically acceptable excipient.

The said excipients are chosen according to the pharmaceutical dosage form and the method of administration desired, from the usual excipients which are known to persons skilled in the art.

In the pharmaceutical compositions of the present invention for oral, sublingual, subcutaneous, intramuscular, intravenous, topical, local, intratracheal, intranasal, transdermal or rectal administration, the active ingredient may be administered in a unit form for administration, mixed with conventional pharmaceutical excipients, to animals and to human beings for the prevention or treatment of type 2 diabetes.

The appropriate unit forms for administration comprise the forms for oral administration such as tablets, soft or hard gelatin capsules, powders, granules and oral solutions or suspensions, the forms for sublingual, buccal, intratracheal, intraocular or intranasal administration, or for administration by inhalation, the forms for topical, transdermal, subcutaneous, intramuscular or intravenous administration, the forms for rectal administration and implants. For topical application, it is possible to use the compounds according to the invention in creams, gels, ointments or lotions.

The forms for oral administration such as gelatin capsules or tablets are preferred.

More particularly, gelatin capsules or tablets are preferred which contain rimonabant at a dose of between 5 and 50 mg, more particularly doses of 10 to 30 mg, in particular the dose of 20 mg.

For use according to the present invention, the rimonabant may be combined with another active ingredient chosen from one of the following therapeutic classes
a hypolipaemic or a hypocholesterolaemic;
another antidiabetic;
another anti-obesity agent.

Thus, the subject of the present invention is also pharmaceutical compositions containing, in combination, an antagonist for the cannabinoid CB₁ receptors, derived from pyrazole, chosen from rimonabant and N-piperidino-5-(4-bromophenyl)-1-(2,4-dichlorophenyl)-4-ethylpyrazole-3-carboxamide, and another active ingredient chosen from one of the following therapeutic classes:
a hypolipaemic or a hypocholesterolaemic;
another antidiabetic.

The expression hypolipaemic or hypocholesterolaemic is understood to mean a compound chosen from fibrates such as alufibrate, beclobrate, bezafibrate, ciprofibrate, clinofibrate, clofibrate, etofibrate, fenofibrate; the statins (HMG-CoA reductase inhibitors), such as atorvastatin, fluvastatin sodium, lovastatin, pravastatin, rosuvastatin, simvastatin, or a compound such as acipimox, aluminum nicotinate, azacosterol, cholestyramine, dextrothyroxine, meglutol, niceritrol, nicoclonate, nicotinic acid, beta-sitosterin, tiadenol.

The expression other antidiabetics is understood to mean a compound belonging to one of the following classes: sulfonylureas, biguanidines, alpha-glucosidase inhibitors, thiazolidinediones, metiglinides, such as acarbose, acetohexamide, carbutamide, chlorpropamide, glibenclamide, glibomuride, gliclazide, glimepiride, glipizide, gliquidone, glisoxepide, glybuzole, glymidine, metahexamide, metformin, miglitol, nateglinide, pioglitazone, repaglinide, rosiglitazone, tolazamide, tolbutamide, voglibose.

According to another particular embodiment, the subject of the present invention is a pharmaceutical composition containing, in combination, rimonabant and metformin, or rimonabant and a sulfonylurea such as acetohexamide, carbutamide, chlorpropamide, glibenclamide, glibomuride, gliclazide, glimepiride, glipizide, tolazamide, tolbutamide, for the treatment of type 2 diabetes.

According to another aspect of the invention, the rimonabant and the other combined active ingredient may be administered simultaneously, separately or spread out over time.

The expression “separate use” is understood to mean the administration, at the same time, of the two compounds of the composition according to the invention, each contained in a distinct pharmaceutical dosage form.

The expression “use spread out over time” is understood to mean the successive administration of the first compound of the composition according to the invention, contained in a pharmaceutical dosage form, and then of the second compound of the composition according to the invention, contained in a distinct pharmaceutical dosage form.

In the case of this “use spread out over time”, the time lapse between the administration of the first compound of the composition according to the invention and the administration of the second compound of the same composition according to the invention generally does not exceed 24 hours, it may be greater if either of the compounds is present in a pharmaceutical formulation allowing, for example, a weekly administration.

The pharmaceutical dosage forms, comprising either only one of the constituent compounds of the composition according to the invention or the combination of the two compounds, which may be used in the various types of uses described above, may for example be appropriate for oral, nasal, parenteral or transdermal administration.

Also, in the case of a “separate use” and of a “use spread out over time”, two distinct pharmaceutical dosage forms may be intended for the same route of administration or for a different route of administration (oral and transdermal or oral and nasal or parenteral and transdermal, and the like).

The invention therefore also relates to a kit containing the rimonabant and another active ingredient or, where appropriate, two combined active ingredients, in which the rimonabant and the said active ingredient, or, where appropriate, two combined active ingredients are in distinct compartments and in similar or different packagings, and are intended to be administered simultaneously, separately or spread out over time.

### EXAMPLE 1

### Action of Rimonabant on Diabetic Patients of the Overweight or Obese Type

The Rio-Diabetes clinical study, carried out over 12 months in 1045 obese subjects with type 2 diabetes treated by monotherapy (metformin or sulfonylureas) compares the effect of rimonabant at the dose of 20 mg versus a placebo product in weight reduction; the improvement of glycosylated haemoglobin (HbAlc), of glycemia, of insulinaemia and lipid parameters. A low-calorie diet (deficit of 600 Kcal/day) is prescribed for all the patients and is introduced 4 weeks before the start of the treatment period.

The subjects treated with rimonabant at the dose de 20 mg for 12 months show a greater weight loss of 4.2±0.4 kg than that observed in the placebo group (p<0.001).

Under rimonabant 20 mg, a difference of 0.7±0.1% is observed in the reduction of the level of HbAlc compared with the placebo (p<0.001). This reduction is maximum at 9 months and is then maintained up to 12 months, whereas the loss of weight appears stabilized after 6 months.

A decrease in glycemia on an empty stomach of 0.64±1.96 mmol/L is observed in the rimonabant 20 mg group, compared with an increase of 0.33±2.32 mmol/L in the placebo group (p<0.001).

For the insulinaemia on an empty stomach, a reduction of 0.7±9.9 μIU/mL is observed under rimonabant 20 mg compared with an increase of 0.4±14.8 μUI/mL in the placebo group (p=0.247).

The insulin resistance is evaluated by the HOMA (Homeostasis Model Assessment) test described by Matthew D. R. et al. in Diabetologica, 1985, 28, 412-419.

An improvement in insulin resistance, evaluated by the HOMA test is objectified under rimonabant 20 mg (−0.5±5.7%) whereas the placebo group induces a deterioration in this insulin resistance.

As regards the lipid profile, an increase in the HDL-c level greater than 8.4±1.2% is observed with rimonabant 20 mg compared with the placebo (p<0.001).

The triglycerides decreased by more than 16.4±3.3% in the treated group compared with the placebo group (p<0.001).

Following analysis of the logistic regression type in which the weight is introduced as a co-variable, an effect independent of the weight loss of about 55% for the improvement in HbAlc and HDL-c and of about 35% for the triglycerides is observed in this study.

Furthermore, in the patients treated with rimonabant at the dose of 20 mg, a reduction in the systolic blood pressure of 0.8±12.8 mmHg (p=0.020) and in the diastolic blood pressure of 1.9±8.2 mmHg (p=0.060) is observed.

Thus, in the subjects treated with rimonabant, the improvement in metabolic parameters such as HbAlc, HDL-c and the triglycerides is not only linked to the weight loss but also to a direct effect of the product.

It is observed that regardless of the antidiabetic treatment received during the study, rimonabant induces a significant weight loss: the difference in weight loss compared with the placebo group is 4.3±0.4 kg (p<0.001) during the rimonabant-metformin combination; it is 3.1±0.5 kg (p<0.001) during the rimonabant-sulfonylurea combination.

It is also observed that the results on HbAlc are similar with an observed difference compared with the placebo of 0.7±0.1% (p<0.001) whether rimonabant is combined with metformin or with a sulfonylurea.

### EXAMPLE 2

### Action of Rimonabant on the Protection of the Pancreas in Obese Rats

The effect of a long-term (12 months) treatment with rimonabant was studied in Zucker rats with established obesity.

The fa/fa strain of obese Zucker rats is characterized by hyperphagia, obesity, dyslipidaemia and type 2 diabetes.

After 12 months, the fa/fa obese Zucker rats treated with the vehicle show a marked hypertrophy of the pancreas (+38%, p<0.05).

This hypertrophy is reversed in a dose-dependent manner by the administration of rimonabant in a dose-dependent manner: +17% and +1% at 3 mg/kg/day and at 10 mg/kg/day (p<0.05), respectively.

### EXAMPLE 3

### Pharmaceutical Composition

For administration to patients, rimonabant is formulated in pharmaceutical compositions which are prepared by wet granulation.

| CONSTITUENTS | | |
|---|---|---|
| Micronized rimonabant | 20.0 | mg |
| Maize starch | 67.50 | mg |
| Lactose monohydrate | 111.66 | mg |
| Povidone * | 5.25 | mg |
| Croscarmellose sodium | 18.75 | mg |
| Sodium lauryl sulfate | 0.34 | mg |
| Microcrystalline cellulose | 75.0 | mg |
| Magnesium stearate | 1.50 | mg |
| Finished tablet at | 300 | mg |

* Povidone is defined in the European Pharmacopoeia as follows: poly(1-(2-oxo-1-pyrrolidinyl)ethylene) and consists of linear 1-vinylpyrrolidin-2-one polymers.

The tablets are preferably coated using an appropriate excipient.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A combination comprising at least one active ingredient chosen from rimonabant and pharmaceutically acceptable salts thereof and at least one second active ingredient chosen from metformin, acetohexamide, carbutamide, chlorpropamide, glibenclamide, glibomuride, gliclazide, glimepiride, glipizide, tolazamide, tolbutamide and pharmaceutically acceptable salts thereof.

2. The combination according to claim 1, wherein said at least one active ingredient and said at least one second active ingredient are administered simultaneously, separately or spread out over time.

3. The combination according to claim 2, wherein said at least one active ingredient and said at least one second active ingredient are administered simultaneously.

4. The combination according to claim 2, wherein said at least one active ingredient and said at least one second active ingredient are administered separately.

5. The combination according to claim 2, wherein said at least one active ingredient and said at least one second active ingredient are administered spread out over time.

6. The combination according to claim 1, wherein said at least second active ingredient is metformin or a pharmaceutically acceptable salt thereof.

7. The combination according to claim 1, wherein said at least second active ingredient is chosen from acetohexamide, carbutamide, chlorpropamide, glibenclamide, glibomuride, gliclazide, glimepiride, glipizide, tolazamide, tolbutamide and pharmaceutically acceptable salts thereof.
